(19) United States
(12) Patent Application Publication
(10) Pub. No.: US 2015/0250448 A1
(43) Pub. Date: Sep. 10, 2015

(54) ULTRASONIC MEASUREMENT APPARATUS AND ULTRASONIC MEASUREMENT METHOD

(71) Applicant: SEIKO EPSON CORPORATION, Tokyo (JP)

(72) Inventor: Natsumi TAMADA, Shiojiri-shi (JP)

(21) Appl. No.: 14/632,306

(22) Filed: Feb. 26, 2015

(30) Foreign Application Priority Data
Mar. 10, 2014 (JP) 2014-046557

(51) Int. Cl. A61B 8/08

(52) U.S. Cl. CPC A61B 8/0891; A61B 8/5223

(57) ABSTRACT

A scanning line immediately above a blood vessel is detected using a received signal of a reflected wave of an ultrasonic wave transmitted to the blood vessel, and candidates for front and rear walls of the blood vessel are detected based on the received signal of the scanning line. Then, vascular front and rear walls pairs of front and rear walls among the candidates are narrowed down, and the narrowed-down vascular front and rear walls pair is regarded as one blood vessel and artery/vein identification is performed for each blood vessel. Measurement of vascular function information is performed for the blood vessel determined to be an artery.

### BACKGROUND

1. Technical Field

The present invention relates to an ultrasonic measurement apparatus that performs measurement using an ultrasonic wave.

2. Related Art

As an example of measuring biological information with an ultrasonic measurement apparatus, the evaluation of a vascular function or the determination of a vascular disease is performed. For example, the intima media thickness (IMT) of the carotid artery, which is an indicator of arteriosclerosis, is measured. In the measurement relevant to the IMT or the like, it is necessary to locate the carotid artery and appropriately determine the measurement point. Typically, the operator places an ultrasonic probe on the neck, locates the carotid artery to be measured while watching a B-mode image displayed on the monitor, and manually sets the found carotid artery as a measurement point.

Although skill is required in order to execute such a series of measurement operations quickly and locate the carotid artery appropriately in the related art, a function to assist the measurement operation has been devised in recent years. For example, JP-A-2008-173177 discloses a method of detecting the vascular wall automatically using the strength of a reflected wave signal from the body tissue, which is obtained by processing the amplitude information of the received reflected wave, and the moving speed of the body tissue, which is obtained by processing the phase information of the received reflected wave. Specifically, a boundary between the vascular wall and the blood flow region is detected based on the first finding that the strength of the reflected wave signal in the blood flow region in the blood vessel is very small compared with the strength of the reflected wave signal in the vascular wall and the second finding that the moving speed calculated from the phase information of the reflected wave signal is high in the blood flow region and low in the vascular wall.

However, in the detection method disclosed in JP-A-2008-173177, a blood vessel can be detected, but it is not possible to determine whether the blood vessel is an artery or a vein. In general, the artery exhibits pulsation, but the vein does not exhibit pulsation. For this reason, the operator tends to simply think that the artery and the vein can be identified by the presence or absence of pulsation. However, in blood vessels relatively close to the heart, such as the internal jugular vein, even veins may exhibit pulsation due to the pressure of the right atrium being transmitted thereto. Therefore, it is difficult to perform correct identification from only the presence or absence of pulsation.

### SUMMARY

An advantage of some aspects of the invention is to realize the technique for identifying the artery and the vein.

A first aspect of the invention is directed to an ultrasonic measurement apparatus including: a transmission and reception control unit that controls transmission of an ultrasonic wave to a blood vessel and reception of the ultrasonic wave that is reflected from the blood vessel; a vessel wall detection unit that detects first and second walls of the blood vessel based on a combination of received signals at different reception timings; and a blood vessel determination unit that determines the blood vessel using a temporal change in a distance between the first and second walls.

As another aspect of the invention, the first aspect of the invention maybe configured as an ultrasonic measurement method including: controlling transmission of an ultrasonic wave to a blood vessel and reception of the ultrasonic wave that is reflected from the blood vessel; detecting first and second walls of the blood vessel based on a combination of received signals at different reception timings; and determining the blood vessel using a temporal change in a distance between the first and second walls.

According to the first aspect and the like of the invention, it is possible to determine a blood vessel, such as an artery, using a distance between the first and second walls of the blood vessel, that is, a temporal change in the blood vessel diameter. The first and second walls of the blood vessel are detected based on the combination of the received signals at different ultrasonic wave reception timings. The movement (pulsation) of the blood vessel is larger than the movement of body tissues around the blood vessel. For this reason, received signals at different reception timings are different depending on whether or not a reflected wave from the first or second wall of the blood vessel is included in the received signal. Thus, it is possible to detect the first and second walls of the blood vessel.

A second aspect of the invention is directed to the ultrasonic measurement apparatus according to the first aspect of the invention, which further includes a unit that detects a position immediately above the blood vessel, at which the ultrasonic wave is transmitted through the blood vessel, using a correlation value obtained by autocorrelation of the combination of the received signals at different positions along the body surface.

According to the second aspect of the invention, a position immediately above the blood vessel, at which the ultrasonic wave is transmitted through the blood vessel, is detected using the correlation value obtained by autocorrelation of the combination of the received signals at different positions along the body surface. Since the movement (pulsation) of the blood vessel is larger than the movement of body tissues around the blood vessel, the correlation value obtained by autocorrelation of the received signals are different depending on whether or not the reflected wave from the first or second wall of the blood vessel is included in the received signal. Therefore, a position immediately above the blood vessel is detected using the correlation value based on the received signal.

A third aspect of the invention is directed to the ultrasonic measurement apparatus according to the first or second aspect of the invention, which further includes a depth position detection unit that detects a depth position of the blood vessel using a correlation value obtained by autocorrelation of the combination of the received signals at different depth positions.

According to the third aspect of the invention, the depth position of the blood vessel is detected using the correlation value obtained by autocorrelation of the combination of the received signals at different depth positions. Since the movement (pulsation) of the blood vessel is larger than the movement of body tissues around the blood vessel, the correlation value obtained by autocorrelation of the received signals are different depending on whether or not the reflected wave from the first or second wall of the blood vessel is included in the received signal. Therefore, the depth position of the blood vessel is detected using the correlation value based on the received signal.

A fourth aspect of the invention is directed to the ultrasonic measurement apparatus according to any one of the first to third aspects of the invention, the blood vessel determination unit may determine a type of the blood vessel using a temporal change in an increasing direction of the distance and a temporal change in a decreasing direction of the distance.

According to the fourth aspect of the invention, the type of the blood vessel is determined using a distance between the first and second walls of the blood vessel, that is, a temporal change in each of the increasing and decreasing directions of the blood vessel diameter. Therefore, for example, even under the specific conditions in which one of the first and second walls hardly moves depending on the state of the body tissues around the blood vessel, it is possible to correctly determine the type of the blood vessel.

A fifth aspect of the invention is directed to the ultrasonic measurement apparatus according to any one of the first to fourth aspects of the invention, which further includes a vascular function measuring unit that performs predetermined vascular function measurement by continuing position measurement with the first and second walls of the blood vessel as tracking targets when the blood vessel is determined to be an artery by the blood vessel determination unit.

According to the fifth aspect of the invention, it is possible to realize a series of processes for automatically locating the artery and performing vascular function measurement for the artery.

### BRIEF DESCRIPTION OF THE DRAWINGS

The invention will be described with reference to the accompanying drawings, wherein like numbers reference like elements.

FIG. 1 is a diagram showing the system configuration of a biological information measuring apparatus.

FIG. 2 is a flowchart showing the flow of the main process performed by an ultrasonic measurement apparatus.

FIG. 3 is an explanatory diagram of an ultrasonic measurement.

FIGS. 4A to 4C are diagrams showing examples of a received signal of a reflected wave.

FIGS. 5A and 5B are explanatory diagrams of the detection of scanning lines immediately above the blood vessel (ultrasonic transducers).

FIGS. 6A to 6C are explanatory diagrams of the detection of a vessel wall depth position candidate.

FIGS. 7A and 7B are diagrams showing examples of the waveform of a change in the blood vessel diameter.

FIGS. 8A to 8C are diagrams showing examples of an artery diameter change rate waveform.

FIGS. 9A to 9C are diagrams showing examples of a vein diameter change rate waveform.

FIG. 10 is a diagram showing the functional configuration of the ultrasonic measurement apparatus.

FIG. 11 is a diagram showing the configuration of a storage unit.

FIG. 12 is a diagram showing an example of the data structure of vascular front and rear walls pair data.

FIG. 13 is a flowchart illustrating the flow of the process of detecting the scanning lines immediately above the blood vessel.

FIG. 14 is a flowchart illustrating the flow of the process of detecting the vessel wall depth position candidate.

FIG. 15 is a flowchart illustrating the process of narrowing down vascular front and rear walls pairs.

FIG. 16 is a flowchart illustrating the flow of the artery determination process.

FIGS. 17A and 17B are diagrams showing examples of a scanning line-signal strength graph.

FIGS. 18A and 18B are explanatory diagrams of the detection of a vessel wall depth position candidate based on the correlation value of the received signal.

### DESCRIPTION OF EXEMPLARY EMBODIMENTS

### System Configuration

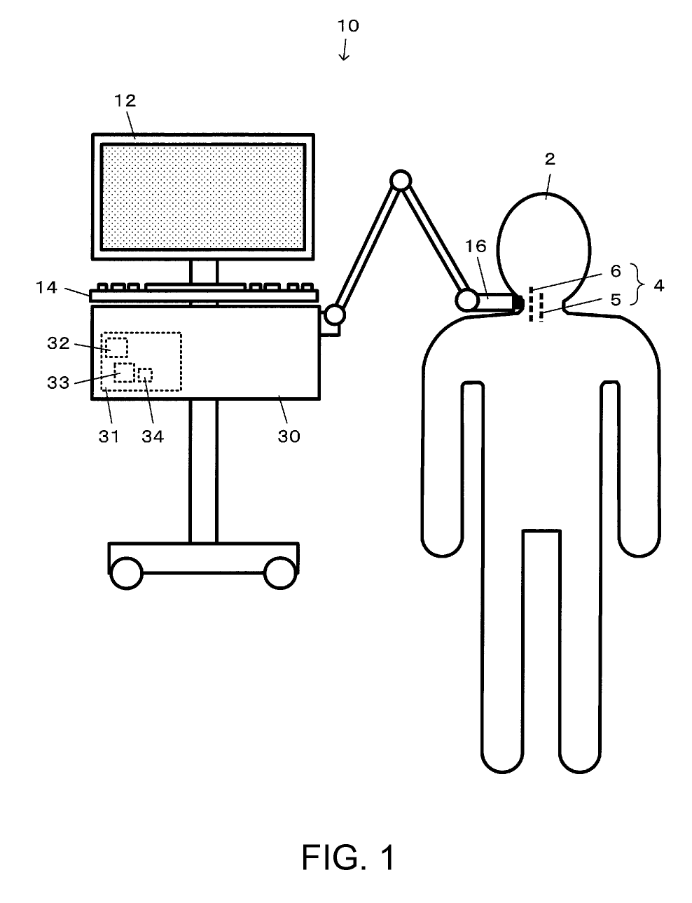

FIG. 1 is a diagram showing an example of the system configuration of an ultrasonic measurement apparatus 10 according to the present embodiment. The ultrasonic measurement apparatus 10 is an apparatus that measures the biological information of a subject 2 by measuring the reflected waves of ultrasonic waves. In the present embodiment, an artery 5 and a vein 6 of blood vessels 4 are automatically identified, and vascular function information, such as the intima media thickness (IMT) of the artery 5, is measured as a piece of biological information.

The ultrasonic measurement apparatus 10 includes a touch panel 12 serving as a unit that displays a measurement result or operation information as an image and as an operation input unit, a keyboard 14 used for operation input, an ultrasonic probe 16, and a processing device 30. A control board 31 is mounted in the processing device 30, and is connected to each unit of the apparatus, such as the touch panel 12, the keyboard 14, and the ultrasonic probe 16, so that signal transmission and reception therebetween are possible.

Not only various integrated circuits, such as a central processing unit (CPU) 32 and an application specific integrated circuit (ASIC), but also a storage medium 33, such as an IC memory or a hard disk, and a communication IC 34 for realizing data communication with an external device are mounted on the control board 31. The processing device 30 realizes various functions according to the present embodiment, such as identification of the arteries and veins, measurement of vascular function information for the identified artery 5, and image display control of the measurement result, including ultrasonic measurement by executing a measurement program stored in the storage medium 33 with the CPU 32 or the like.

Specifically, by the control of the processing device 30, the ultrasonic measurement apparatus 10 transmits an ultrasonic beam from the ultrasonic probe 16 to the subject 2 and receives the reflected wave. Then, by performing amplification and signal processing on a received signal of the reflected wave, it is possible to generate reflected wave data, such as a temporal change or position information of the structure in the living body of the subject 2. Images of respective modes of so-called A mode, B mode, M mode, and color Doppler are included in the reflected wave data. Measurement using an ultrasonic wave is repeatedly performed at predetermined periods. The measurement unit is referred to as a “frame”.

By setting a region of interest (tracking point) in the reflected wave data as a reference, the ultrasonic measurement apparatus 10 can perform so-called “tracking” that is tracking each region of interest between different frames and calculating the displacement.

First, the overview of the process leading up to the measurement of vascular function information will be described.

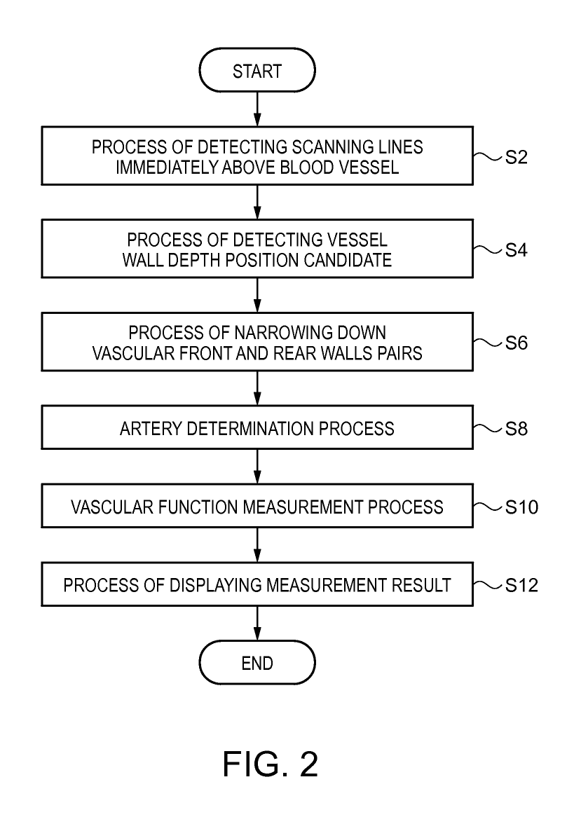

FIG. 2 is a flowchart showing the flow of the main process performed by the ultrasonic measurement apparatus 10. It is assumed that the ultrasonic probe 16 is placed toward the carotid artery by the operator. The ultrasonic measurement apparatus 10 detects an ultrasonic transducer (can also be referred to as a scanning line instead of the transducer) located immediately above the blood vessel regardless of the distinction of arteries and veins (step S2). This is referred to as a “scanning line immediately above the blood vessel”. In addition, “immediately above” referred to herein, needless to say, includes a position directly above the blood vessel center literally, but also has the meaning of allowing a slight shift in a radial direction from the position immediately above in a range that is sufficient to measure the vascular function information of interest. “immediately above” or “directly above” does not necessarily mean an opposite direction to a vertical direction (opposite direction to the gravity), but is the meaning in terms of the operation of the operator who handles the ultrasonic probe 16 to place the ultrasonic probe 16 “immediately above” or “directly above” the blood vessel on the body surface. Therefore, a position (a transducer or a scanning line) where an ultrasonic wave is transmitted through a blood vessel can also be referred to as a scanning line immediately above the blood vessel.

Then, a candidate at a depth position that seems to be a vascular wall is detected from the reflected wave data in the scanning lines immediately above the blood vessel (step S4). Although a part regarded as the front wall of (vascular wall facing the skin side) the blood vessel or the rear wall (vascular wall located opposite the front wall) of the blood vessel is detected in this stage, a body part other than the blood vessels may be included in depth position candidates since the part has not yet been determined as a blood vessel. Therefore, the ultrasonic measurement apparatus 10 narrows down the pairs of front and rear walls of the blood vessels from the detected depth position candidates (step S6). The narrowed-down pair of depth position candidates is called a “vascular front and rear walls pair”. One of the front and rear walls of the blood vessel is a first wall, and the other is a second wall.

Then, the ultrasonic measurement apparatus 10 performs artery determination for each narrowed-down vascular front and rear walls pair, thereby identifying whether the vascular front and rear walls pair corresponds to the artery or corresponds to the vein (step S8). For the vascular front and rear walls pair determined to be the artery 5, the ultrasonic measurement apparatus 10 performs vascular function measurement (step S10). Then, the measurement result is displayed on the touch panel 12 (step S12). The content of the vascular function measurement may be other content without being limited to the IMT, and a known technique can be appropriately used.

### Description of Principle

Next, each step will be described in detail.

First, a step of detecting the scanning lines immediately above the blood vessel will be described. The detection of the scanning lines immediately above the blood vessel is based on the movement of body tissues. That is, a blood vessel position is determined based on the finding that blood vessels move largely periodically with the beating of the heart but the movement of other body tissues around the blood vessels is small compared with the movement of the blood vessels.

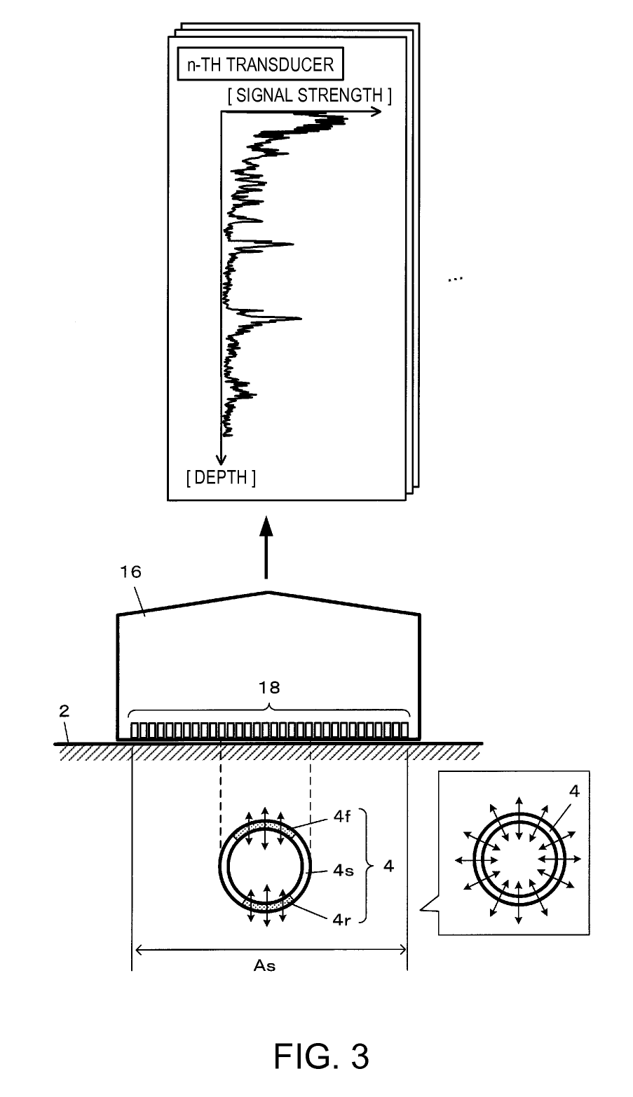

FIG. 3 is a diagram schematically showing a state where the ultrasonic probe 16 is in contact with the body surface of the subject 2 in order to perform ultrasonic measurement, and is a diagram showing the cross-section of the blood vessel 4 in a short-axis direction. A plurality of ultrasonic transducers 18 are built into the ultrasonic probe 16. In the example shown in FIG. 3, one ultrasonic beam is irradiated from each ultrasonic transducer 18 toward the bottom from the top in the diagram. The range covered by the ultrasonic transducer 18 is a probe scanning range As. The ultrasonic transducers 18 may be provided in a plurality of columns in a depth direction toward the diagram, that is, may be provided in a planar shape. Alternatively, the ultrasonic transducers 18 may be provided only in a horizontal direction with only one column in the depth direction toward the diagram.

The blood vessel 4 repeats approximately isotropic expansion/contraction by the beating (expansion/contraction) of the heart. Therefore, a stronger reflected wave can be received as the area of the surface perpendicular to the direction of the ultrasonic beam becomes larger. However, as a reflected wave becomes more parallel to the beam direction, it becomes more difficult to receive the reflected wave. For this reason, in the ultrasonic measurement, the reflected wave from a front wall 4f and a rear wall 4r of the blood vessel 4 is detected strongly, but the reflected wave from a lateral wall 4s is weak. In other words, if there is the blood vessel 4 in the probe scanning range As, a strong reflected wave relevant to the front and rear walls appears in the reflected wave signal at the position of the ultrasonic transducer 18 located immediately above the blood vessel 4.

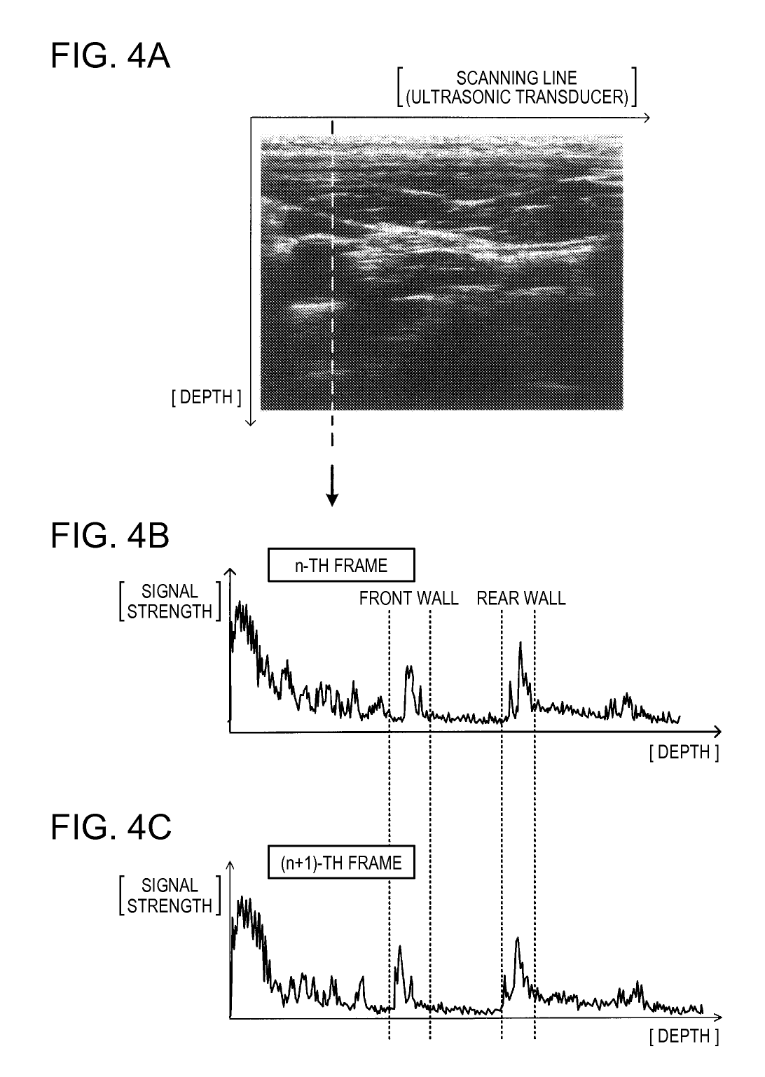

FIGS. 4A to 4C are graphs showing examples of the received signal of the reflected wave at the position of a certain ultrasonic transducer 18. FIG. 4A is an image obtained by converting the received signal strength in each ultrasonic transducer 18 into a brightness, that is, a B-mode image. The horizontal axis indicates an arrangement direction of the ultrasonic transducers 18, that is, a scanning line direction, and the vertical axis indicates a depth direction. FIG. 4B is a “depth-signal strength graph” showing a measurement result in the first frame of the measurement period, and FIG. 4C is a “depth-signal strength graph” showing a measurement result in the second frame of the measurement period.

If there is the blood vessel 4 in a transmission direction (in FIG. 4A, a direction indicated by a dashed line) of the ultrasonic wave from the ultrasonic transducer 18, a strong reflected wave relevant to the front and rear walls is detected. Also in FIGS. 4B and 4C, peaks of two strong reflected waves that can be clearly identified appear at positions deeper than the group of reflected waves near the body surface.

When the signal strengths at respective depths are compared between frames, a change in the signal strength occurs at depth positions corresponding to the front and rear walls of the blood vessel. This is because the blood vessels pulsate. In addition, a slight signal strength change occurs because body tissues other than the blood vessel are also moved slightly due to the influence of pulsation or the like. However, this is not as large as the change for the blood vessel (specifically, front and rear walls of the blood vessel).

In the present embodiment, even if a characteristic indicating a blood vessel (front and rear walls) appears in the signal strength in a certain ultrasonic transducer 18, it is not determined immediately that the ultrasonic transducer 18 is located immediately above the blood vessel. Instead, the ultrasonic transducer 18 located immediately above the blood vessel, that is, a scanning line immediately above the blood vessel is determined from the degree of change in the received signal strength by performing an autocorrelation operation of the received signal strength between different frames.

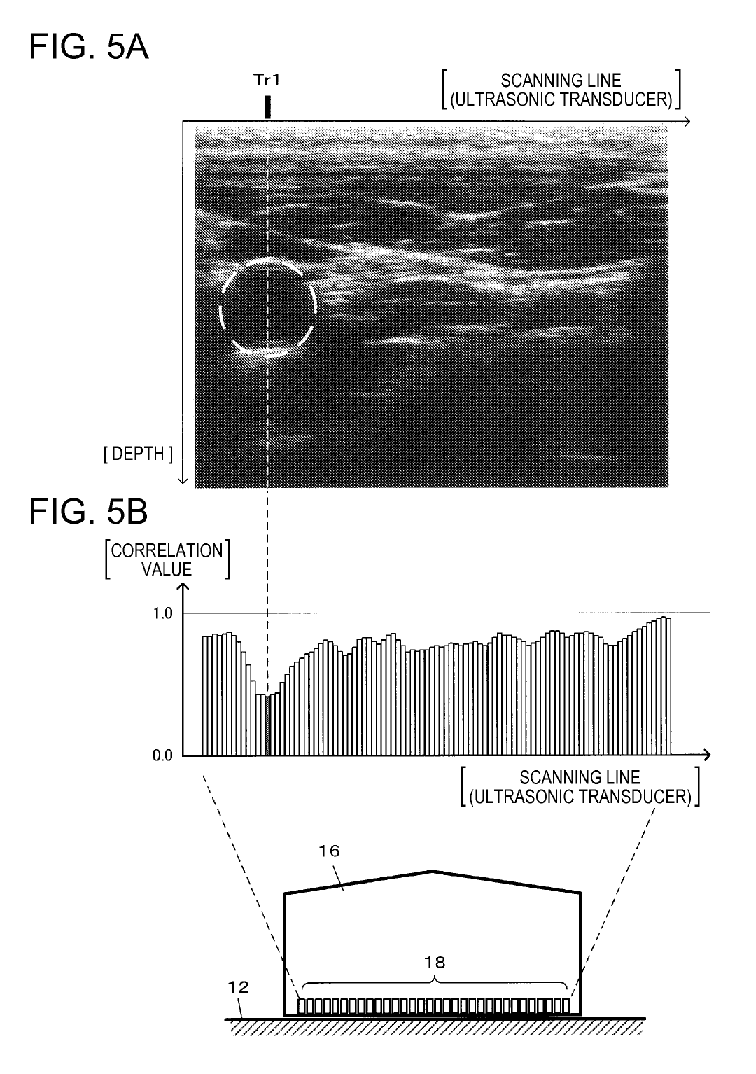

FIGS. 5A and 5B are diagrams illustrating the detection of scanning lines immediately above the blood vessel based on the correlation operation of the received signal strength between frames. FIG. 5A is a B-mode image, and FIG. 5B is a graph showing the correlation value of the received signal strength in each ultrasonic transducer 18 between two consecutive frames. The correlation value is a value normalized in the range of “0.0 to 1.0”, and 1.0 indicates the same.

When there is no blood vessel below the ultrasonic transducer 18, a correlation value of the received signal between two consecutive frames in a certain ultrasonic transducer 18 is a large value. This is because there is no movement or little movement of body tissues over time and accordingly there is no change in the signal strength at each depth position or the change in the signal strength at each depth position is small even if there is a change. On the other hand, when there is a blood vessel, the correlation value is small because the depth positions of the front and rear walls of the blood vessel are changed by pulsation. That is, as shown in the graph of FIG. 5B, the ultrasonic transducer 18 located closer to the position immediately above the blood vessel center has a smaller correlation value. Accordingly, an ultrasonic transducer having a correlation value satisfying predetermined conditions is determined to be the ultrasonic transducer 18 located immediately above the blood vessel, that is, a scanning line immediately above the blood vessel. More specifically, in the graph of FIG. 5B, the ultrasonic transducer 18 corresponding to the minimum value is determined to be a scanning line immediately above the blood vessel. In the example shown in FIGS. 5A and 5B, an ultrasonic transducer Tr1 corresponds to this.

Next, a step of detecting a vessel wall depth position candidate will be described.

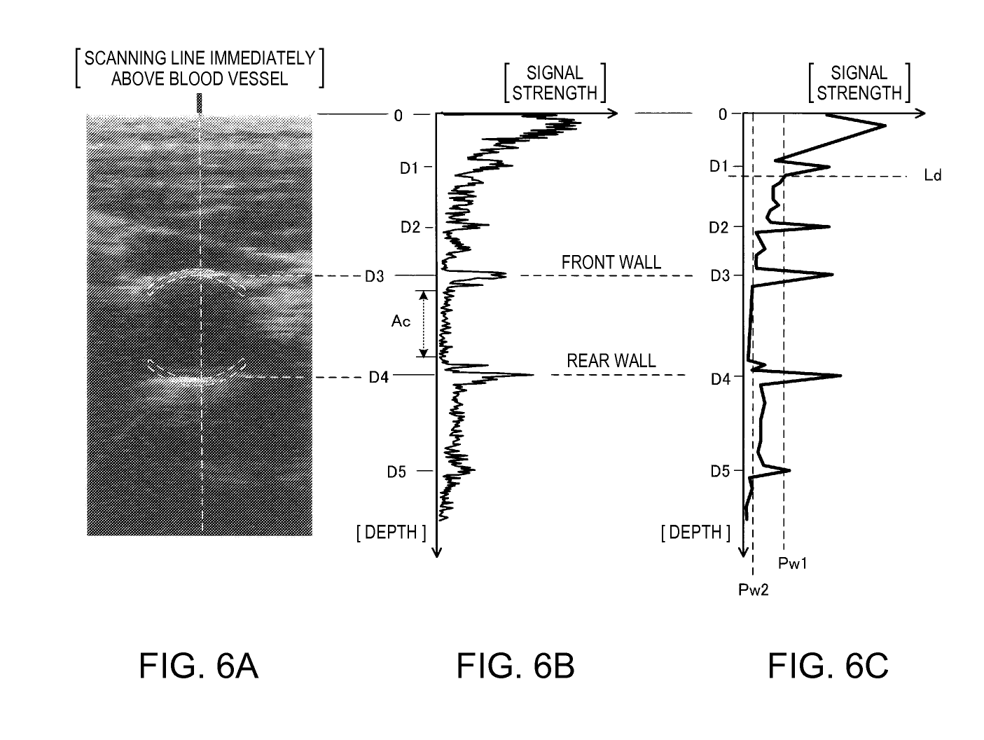

FIGS. 6A to 6C are diagrams for explaining the principle of the detection of a vessel wall depth position candidate. FIG. 6A is a B-mode image of a blood vessel part, FIG. 6B is a signal strength graph of the received signal of the reflected wave in the scanning lines immediately above the blood vessel, and FIG. 6C is a graph obtained by smoothing changes in the signal strength more clearly.

First, peaks, in which signal strengths equal to or higher than a predetermined vessel wall equivalent signal level Pw1 are obtained, are extracted. In this case, a strong reflected wave equal to or higher than the vessel wall equivalent signal level Pw1 is obtained from the front and rear walls of the blood vessel, but a strong reflected wave may also be similarly obtained from the surrounding tissues. For this reason, a plurality of peaks (in FIGS. 6A to 6C, five peaks D1 to D5) may appear in the signal strength graph. Therefore, the peaks are narrowed down based on the likelihood of the vascular wall.

In the narrowing down, first, a peak of a shallower position than the minimum reference depth Ld is excluded from the plurality of peaks D1 to D5. The minimum reference depth Ld is the limit of shallowness at which a blood vessel having an appropriate size as a measurement target can be present, and a value larger than at least the dermis is set as the minimum reference depth Ld. In the example shown in FIGS. 6A to 6C, the peak D1 is excluded from the vessel wall depth position candidates since the depth of the peak D1 is less than the minimum reference depth Ld.

Then, the peaks are narrowed down based on the finding that the signal strength of the reflected wave of the intravascular lumen is very low compared with the surrounding tissues. That is, the peaks of the signal strength regarded as the vessel wall depth position candidates are determined as a pair of front and rear walls, and are temporarily combined. Then, the signal strengths between the respective combinations are statistically processed to calculate an average value or a median. Then, a combination satisfying the vascular front and rear walls pair equivalent conditions of “combination in which the statistical processing value is less than a predetermined intravascular lumen equivalent signal level Pw2” and “combination in which another peak is not present between the combined peaks” is extracted, and this is set as a “front and rear walls pair”.

For example, in the example shown in FIG. 6C, a combination in which the peak D4 is regarded as the front wall and the peak D5 is regarded as the rear wall is excluded since the statistical processing value of the signal strength between the two peaks exceeds the intravascular lumen equivalent signal level Pw2. In addition, a combination in which the peak D3 is regarded as the front wall and the peak D5 is regarded as the rear wall and a combination in which the peak D2 is regarded as the front wall and the peak D4 is regarded as the rear wall are also excluded since another peak is present between peaks. On the other hand, a combination in which the peak D3 is regarded as the front wall and the peak D4 is regarded as the rear wall satisfies the conditions described above. Accordingly, this combination is regarded as a “front and rear walls pair”.

As a method of narrowing down, focusing on the finding that the vascular wall shows a larger movement than the surrounding tissues, determination may be made from the displacement in one cardiac cycle of the peak position of the signal strength difference between frames. In the narrowing down method, however, for example, in a situation where there is almost no movement at the position of the front wall or the rear wall of the blood vessel in the positional relationship between the blood vessel 4 and the surrounding tissues, it is not possible to correctly narrow down the vascular front and rear walls pairs. However, according to the narrowing down method of the present embodiment, it is possible to reliably identify the vascular front and rear walls pair even in such a situation.

Next, an artery determination step will be described.

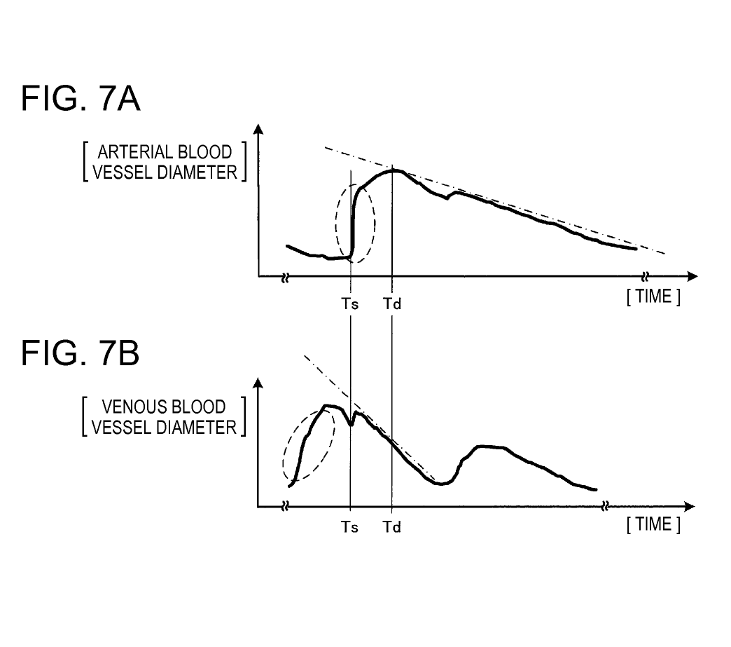

FIGS. 7A and 7B are graphs showing examples of a change in the blood vessel diameter for approximately one beat of the cardiac cycle, where FIG. 7A is a graph of the arterial blood vessel diameter and FIG. 7B is a graph of the venous blood vessel diameter.

The vascular wall of the artery has a structure with high stretchability and elasticity so as to be able to withstand a pulsatile blood flow, which flows from the heart, and the blood pressure. For this reason, the blood vessel diameter increases rapidly during systole (Ts) according to the beating of the heart, and decreases slowly during diastole (Td) to return to the original thickness. Therefore, since the blood vessel diameter increases rapidly immediately after systole (Ts), the graph of the arterial blood vessel diameter rises abruptly (for example, a portion surrounded by the dashed line in FIG. 7A). On the other hand, since the blood vessel diameter decreases slowly during diastole (Td), the graph falls gently. Thus, in the case of the artery, the degree of change in a direction in which the blood vessel diameter increases is larger than that in a direction in which the blood vessel diameter decreases, and the difference is noticeable.

On the other hand, the vascular wall (vein wall) of the vein is thinner than the vascular wall (artery wall) of the artery. For this reason, the vascular wall (vein wall) of the vein has poor elasticity. In addition, blood pressure applied to the vein wall is lower than the blood pressure applied to the artery wall. Therefore, in the case of the vein, when the degree of change in the rise (a portion surrounded by the dashed line in FIG. 7B) of the graph in a direction in which the blood vessel diameter increases is compared with the degree of change in the lowering of the graph in which the blood vessel diameter decreases, the difference as in the case of the artery does not appear.

In the present embodiment, the difference in the displacement characteristics of the vascular wall due to pulsation between the artery and the vein is identified using the displacement rate waveform of the vascular wall, and is used for artery determination. Specifically, a temporal change in the distance between the front and rear walls, that is, the rate of change in the blood vessel diameter (hereinafter, referred to as a “diameter change rate”) is calculated by setting the position regarded as the vascular front and rear walls pair as a region of interest and calculating the displacement rate of the vascular wall from the amount of displacement per unit time using the tracking function for tracking each region of interest between different frames. Then, the artery/vein is identified from the ratio between the extreme value of a temporal change in the diameter change rate in the direction of diameter increase and the extreme value of a temporal change in the diameter change rate in the direction of diameter decrease.

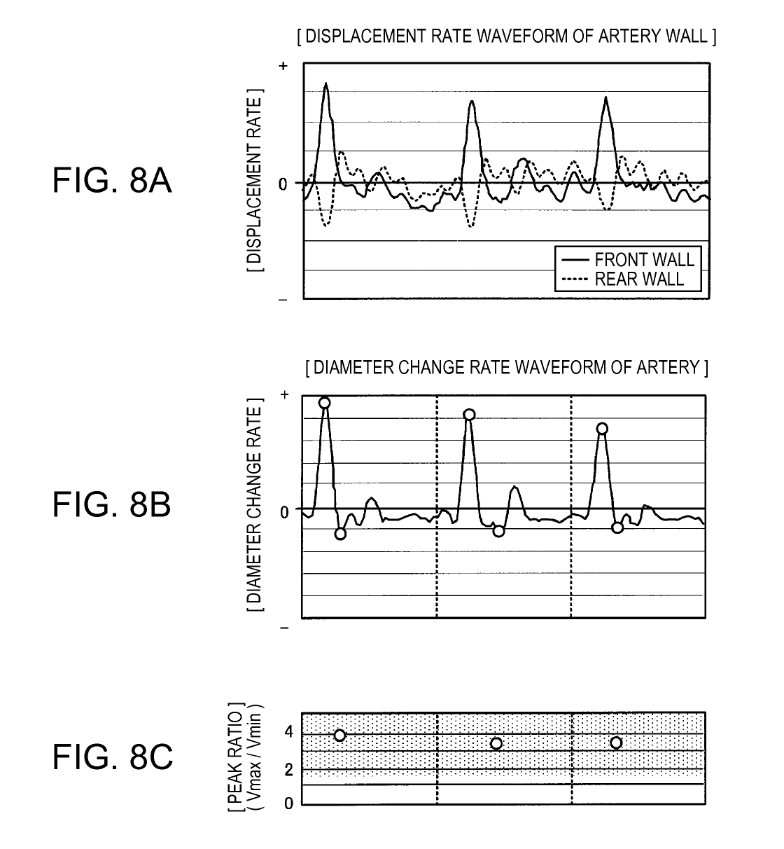

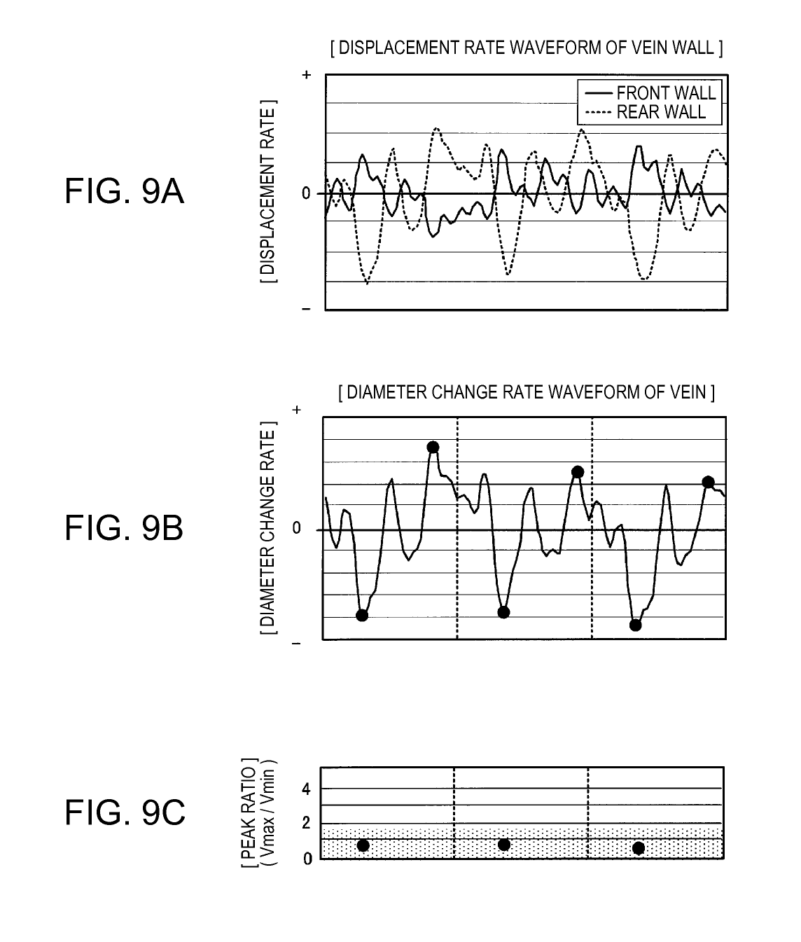

For example, FIG. 8A is a diagram showing a displacement rate waveform of the artery wall for approximately three beats of the cardiac cycle, FIG. 8B is a diagram showing an artery diameter change rate waveform for approximately three beats of the cardiac cycle, and FIG. 8C is a diagram showing the ratio between the absolute values of extreme values (maximum and minimum values), that is, the peak ratio (maximum value/minimum value) in the diameter change rate waveform. For example, FIG. 9A is a diagram showing a displacement rate waveform of the vein wall for approximately three beats of the cardiac cycle, FIG. 9B is a diagram showing a vein diameter change rate waveform for approximately three beats of the cardiac cycle, and FIG. 9C is a diagram showing the ratio between the absolute values of extreme values (maximum and minimum values), that is, the peak ratio (maximum value/minimum value) in the diameter change rate waveform.

The difference between the displacement characteristics of the artery wall, in which the difference between the rate of change in a direction in which the blood vessel diameter increases and the rate of change in a direction in which the blood vessel diameter decreases is noticeable, and the displacement characteristics of the vein wall, in which the difference between the rate of change in a direction in which the blood vessel diameter increases and the rate of change in a direction in which the blood vessel diameter decreases is smaller than that in the case of the artery wall, is expressed as a difference in the peak ratio, as shown in FIG. 8C and FIG. 9C.

More specifically, the peak ratio based on the artery diameter change rate waveform is relatively high, and the peak ratio based on the vein diameter change rate waveform is relatively low. The boundary is generally in the range of “1.4” to “1.6”. In the present embodiment, artery/vein identification is performed by using the intermediate value “1.5” as a threshold value of the peak ratio when the blood vessel is an artery. The threshold value, needless to say, can be appropriately set depending on age range, race, sex, medical history, or the like of the subject.

### Description of Functional Configuration

Next, the functional configuration for realizing the present embodiment will be described.

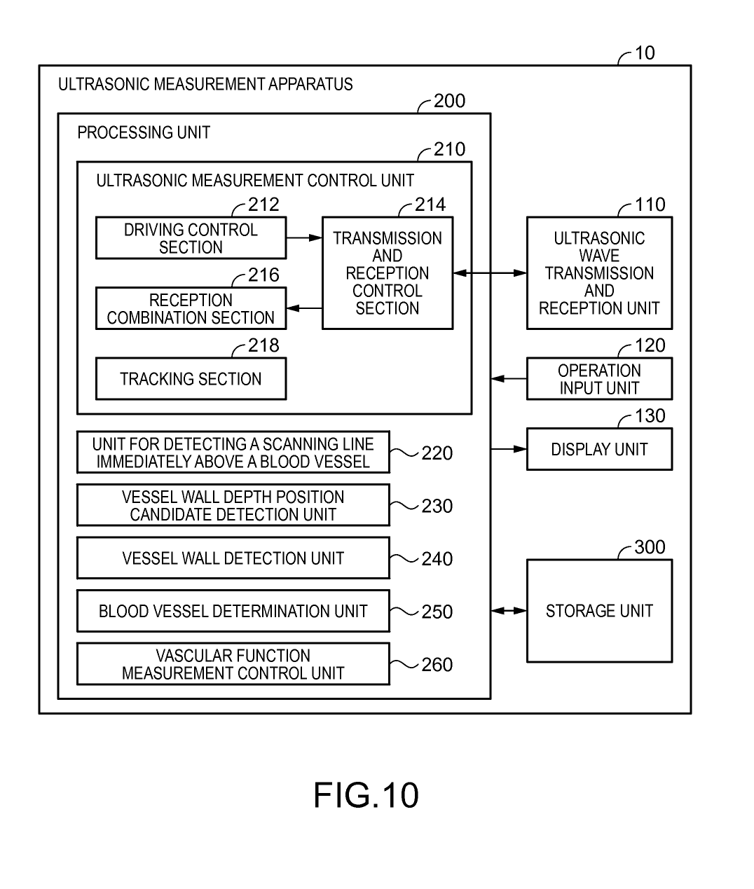

FIG. 10 is a block diagram showing an example of the functional configuration of the ultrasonic measurement apparatus 10 according to the present embodiment. The ultrasonic measurement apparatus 10 includes an ultrasonic wave transmission and reception unit 110, an operation input unit 120, a display unit 130, a processing unit 200, and a storage unit 300.

The ultrasonic wave transmission and reception unit 110 transmits an ultrasonic wave with a pulse voltage output from the processing unit 200. Then, the ultrasonic wave transmission and reception unit 110 receives a reflected wave of the transmitted ultrasonic wave, converts the reflected wave into a reflected wave signal, and outputs the reflected wave signal to the processing unit 200. The ultrasonic probe 16 shown in FIG. 1 corresponds to the ultrasonic wave transmission and reception unit 110.

The operation input unit 120 receives various kinds of operation input by the operator, and outputs an operation input signal corresponding to the operation input to the processing unit 200. The operation input unit 120 can be implemented by a button switch, a lever switch, a dial switch, a track pad, a mouse, or the like. In the example shown in FIG. 1, the touch panel 12 or the keyboard 14 corresponds to the operation input unit 120.

The display unit 130 is realized by a display device, such as a liquid crystal display (LCD), and performs various kinds of display based on the display signal from the processing unit 200. In FIG. 1, the touch panel 12 corresponds to the display unit 130.

The processing unit 200 is realized by a microprocessor, such as a CPU or a graphics processing unit (GPU), or an electronic component, such as an ASIC or an IC memory, for example. In addition, the processing unit 200 performs control of the input and output of data to each functional unit, and calculates biological information of the subject 2 by performing various kinds of arithmetic processing based on a predetermined program or data, the operation input signal from the operation input unit 120, the reflected wave signal from the ultrasonic wave transmission and reception unit 110, or the like. The processing device 30 and the control board 31 shown in FIG. 1 correspond to the processing unit 200.

In the present embodiment, the processing unit 200 includes an ultrasonic measurement control unit 210, a unit for detecting a scanning line immediately above a blood vessel 220, a vessel wall depth position candidate detection unit 230, a vessel wall detection unit 240, a blood vessel determination unit 250, and a vascular function measurement control unit 260.

The ultrasonic measurement control unit 210 controls the transmission of an ultrasonic wave toward the blood vessel and the reception of a reflected wave. For example, the ultrasonic measurement control unit 210 includes a driving control section 212, a transmission and reception control section 214, a reception combination section 216, and a tracking section 218, and performs overall control of ultrasonic measurement. The ultrasonic measurement control unit 210 can be realized by known techniques.

The driving control section 212 controls the transmission timing of ultrasonic pulses from the ultrasonic probe 16, and outputs a transmission control signal to the transmission and reception control section 214.

The transmission and reception control section 214 generates a pulse voltage according to the transmission control signal from the driving control section 212, and outputs the pulse voltage to the ultrasonic wave transmission and reception unit 110. In this case, it is possible to adjust the output timing of the pulse voltage to each ultrasonic transducer by performing transmission delay processing. In addition, it is possible to perform amplification or filtering of the reflected wave signal output from the ultrasonic wave transmission and reception unit 110 and to output the result to the reception combination section 216.

The reception combination section 216 performs processing relevant to the so-called focus of a received signal by performing delay processing as necessary, thereby generating reflected wave data 320.

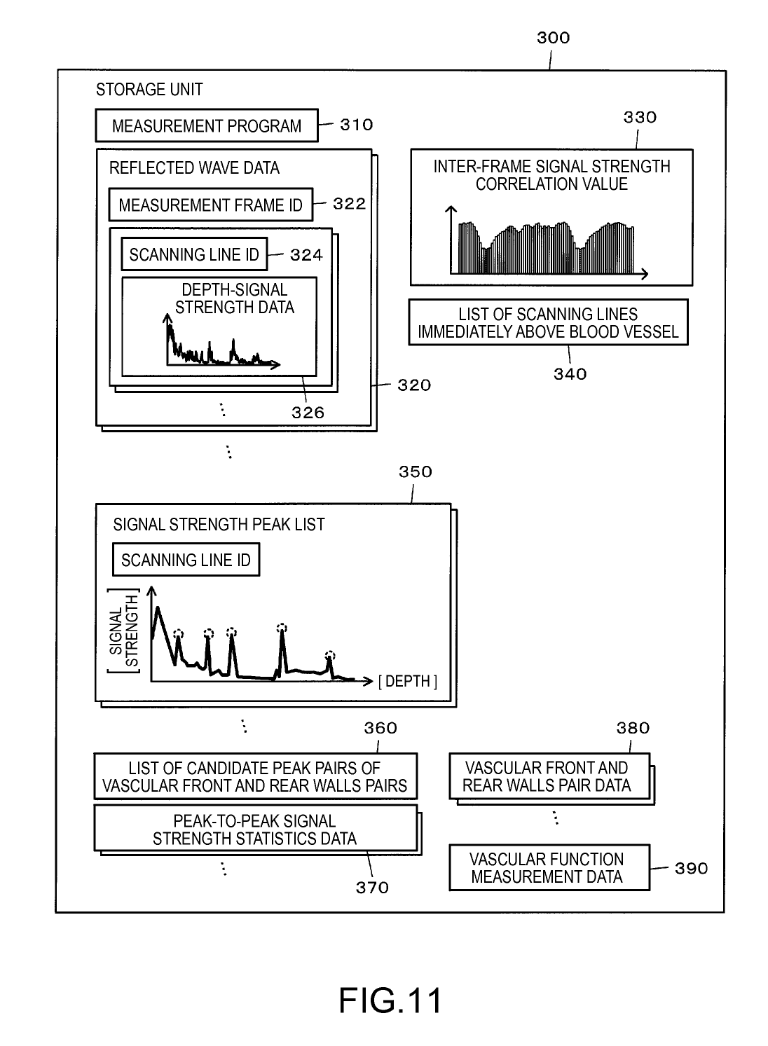

As shown in FIG. 11, the reflected wave data 320 is generated for each frame. A piece of reflected wave data 320 includes a corresponding measurement frame ID 322, scanning line ID 324, and depth-signal strength data 326 corresponding thereto.

The tracking section 218 performs processing relevant to so-called “tracking” that is for tracking the position of a region of interest between frames of ultrasonic measurement based on the reflected wave data (reflected wave signal). For example, it is possible to perform processing for setting a region of interest (tracking point) in the reflected wave data (for example, a B-mode image) as a reference, processing for tracking each region of interest between different frames, and processing for calculating the displacement for each region of interest. Functions, such as so-called “echo tracking” or “phase difference tracking” that is known, are realized.

The unit for detecting a scanning line immediately above a blood vessel 220 performs arithmetic processing for detecting the scanning lines immediately above the blood vessel or controls each unit. That is, control relevant to the above-described step of detecting the scanning lines immediately above the blood vessel is performed (refer to FIGS. 5A and 5B). In the detection of a scanning line immediately above the blood vessel, when measurement for two consecutive frames is performed, the correlation value of the reflected wave received signal (depth-signal strength data 326) between the two frames is calculated for all ultrasonic transducers, and is stored as an inter-frame signal strength correlation value 330. Then, the ultrasonic transducer (scanning line) having a correlation value that is a minimum value and is equal to or less than a predetermined reference value is detected as a scanning line immediately above the blood vessel. The scanning line immediately above the blood vessel and the detected scanning line ID are stored as a list of scanning lines immediately above a blood vessel 340.

As a method of calculating the inter-frame signal strength correlation value 330, another method may be used. For example, the calculation of a correlation value based on a correlation operation (autocorrelation operation), which is performed whenever ultrasonic measurement for two consecutive frames is performed, may be repeated for each ultrasonic transducer, and the average value or the median of the correlation values for a predetermined amount of time (predetermined number of frames) maybe set as the inter-frame signal strength correlation value 330.

The vessel wall depth position candidate detection unit 230 detects a depth position regarded as a vessel wall based on the received signal of the reflected wave in the scanning lines immediately above the blood vessel. A part of control relevant to the above-described step of detecting the vascular wall depth position candidate is performed (refer to FIGS. 6A to 6C). In the detection of a vessel wall depth position candidate, a depth position candidate regarded as a vascular wall, that is, a peak of the signal strength, is extracted from the reflected wave received signal (depth-signal strength data 326) of the scanning line for each scanning line immediately above the blood vessel, thereby generating a signal strength peak list 350.

The vessel wall detection unit 240 detects the front and rear walls of the blood vessel using the received signal of the reflected wave in the scanning lines immediately above the blood vessel. A part of control relevant to the above-described step of narrowing down the front and rear walls pair of the blood vessel is performed (refer to FIG. 6C). In the detection of front and rear walls of the blood vessel, a combination of the peak assumed to be a front wall and the peak assumed to be a rear wall is generated from the peaks of the signal strength stored in the signal strength peak list 350, that is, from the depth position candidates regarded as vascular walls, and this is stored as the list of candidate peak pairs of vascular front and rear walls pairs 360. Then, a statistical value of the signal strength between the peaks of the pair is calculated for each pair of peaks assumed to be front and rear walls that has been generated, and this is stored as peak-to-peak signal strength statistics data 370. In addition, for each pair of peaks, a pair in which the statistical value of the signal strength between the peaks of the pair satisfies the vascular front and rear walls pair equivalent conditions is narrowed down, and the pair is detected as a “front and rear walls pair”.

The blood vessel determination unit 250 determines the type of the artery/vein using a temporal change in the distance between the front and rear walls. A part of control relevant to the artery determination step described above is performed (refer to FIGS. 7A to 9C).

When the blood vessel determination unit 250 determines that the blood vessel is an artery, the vascular function measurement control unit 260 performs control relevant to predetermined vascular function measurement by continuing position measurement with the front and rear walls of the blood vessel as a tracking target.

The storage unit 300 is realized by a storage medium, such as an IC memory, a hard disk, or an optical disc, and stores various programs or various kinds of data, such as data in the operation process of the processing unit 200. In FIG. 1, the storage medium 33 mounted in the control board 31 of the processing device 30 corresponds to the storage unit 300. In addition, the connection between the processing unit 200 and the storage unit 300 is not limited to a connection using an internal bus circuit in the apparatus, and may be realized by using a communication line, such as a local area network (LAN) or the Internet. In this case, the storage unit 300 may be realized by using a separate external storage device from the ultrasonic measurement apparatus 10.

In addition, as shown in FIG. 11, the storage unit 300 stores a measurement program 310, the reflected wave data 320, the inter-frame signal strength correlation value 330, the list of scanning lines immediately above a blood vessel 340, the signal strength peak list 350, the list of candidate peak pairs of vascular front and rear walls pairs 360, the peak-to-peak signal strength statistics data 370, vascular front and rear walls pair data 380, and vascular function measurement data 390. Needless to say, frame identification information, various flags, counter values for time checking, and the like other than those described above can also be appropriately stored.

The processing unit 200 realizes the functions of the ultrasonic measurement control unit 210, the unit for detecting a scanning line immediately above a blood vessel 220, the vessel wall depth position candidate detection unit 230, the vessel wall detection unit 240, the blood vessel determination unit 250, the vascular function measurement control unit 260, and the like by reading and executing the measurement program 310. In addition, when realizing these functional units with hardware, such as an electronic circuit, a part of the program for realizing the function can be omitted.

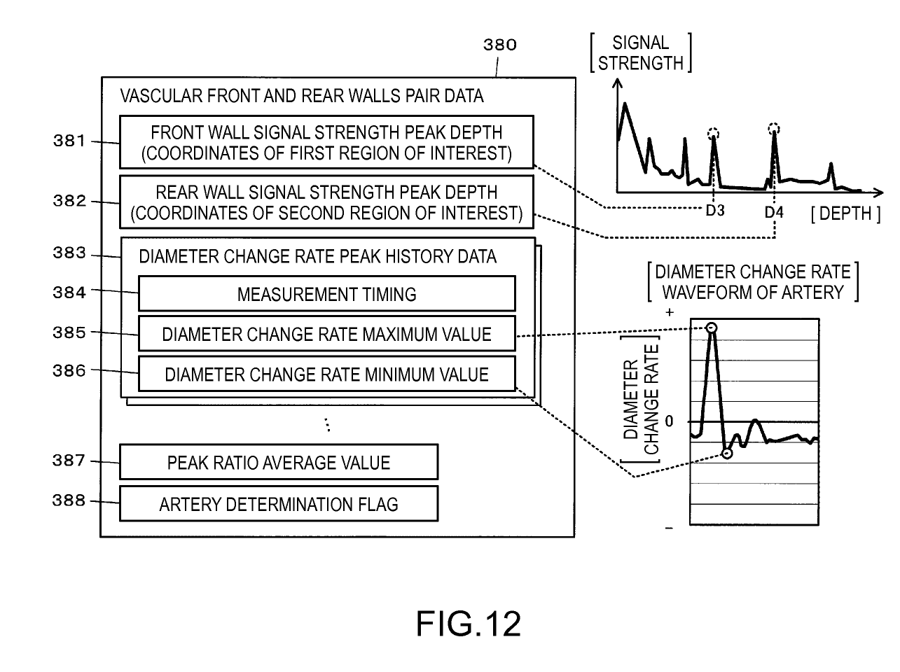

FIG. 12 is a diagram showing an example of the data structure of the vascular front and rear walls pair data 380. The vascular front and rear walls pair data 380 is generated for each vascular front and rear walls pair, and includes a front wall signal strength peak depth 381, a rear wall signal strength peak depth 382, diameter change rate peak history data 383, a peak ratio average value 387, and an artery determination flag 388.

The front wall signal strength peak depth 381 and the rear wall signal strength peak depth 382 are depth positions of the peaks of the signal strengths regarded as front and rear walls, and correspond to the coordinates of a first region of interest and the coordinates of a second region of interest in the tracking control for artery determination, respectively.

In the diameter change rate peak history data 383, an extreme value of the diameter change rate waveform in one beat of the cardiac cycle of the blood vessel having the vascular front and rear walls pair is stored. In a piece of diameter change rate peak history data 383, for example, measurement timing 384, a diameter change rate maximum value 385 of the blood vessel diameter, and a diameter change rate minimum value 386 of the blood vessel diameter are stored.

In the peak ratio average value 387, the average value of the peak ratio (=diameter change rate maximum value 385/diameter change rate minimum value 386) calculated for each piece of the diameter change rate peak history data 383 is further stored.

The artery determination flag 388 is a flag in which “1” is stored when determination as an artery is made.

### Description of the Flow of the Process

Next, the operation of the ultrasonic measurement apparatus 10 in each step from the detection of the scanning lines immediately above the blood vessel to the artery determination processing will be described (refer to FIG. 2).

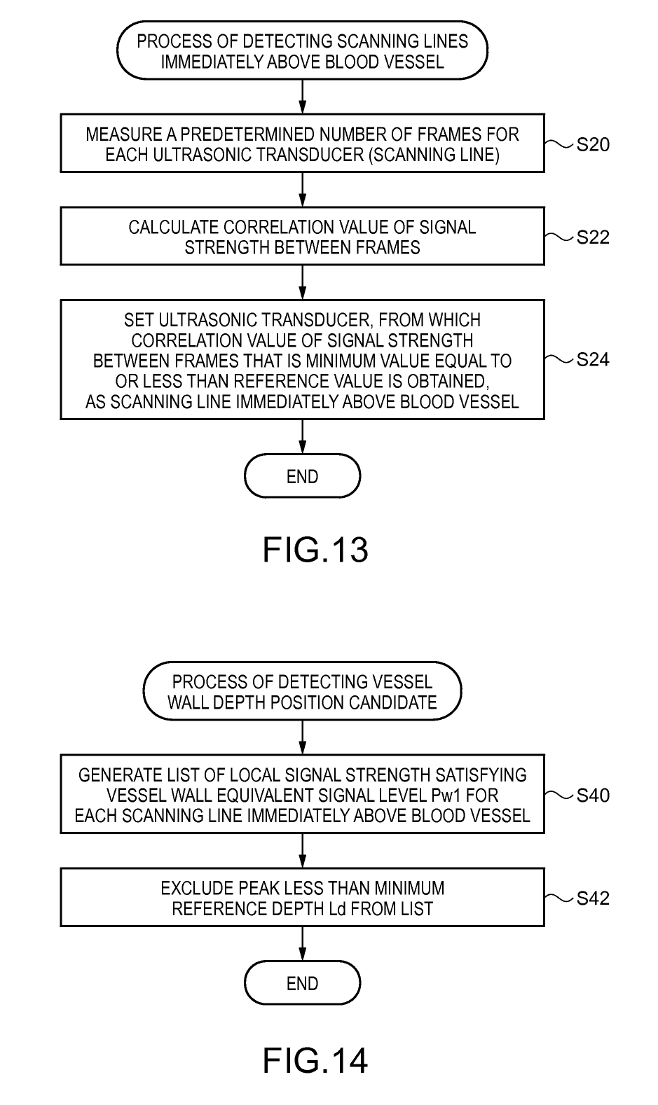

FIG. 13 is a flowchart for explaining the flow of the process of detecting the scanning lines immediately above the blood vessel in the ultrasonic measurement apparatus 10 according to the present embodiment.

In this process, first, the processing unit 200 transmits ultrasonic beams of a predetermined number of frames to each ultrasonic transducer (scanning line) and receives the reflected waves (step S20). Then, the reflected wave data 320 (refer to FIG. 11) is stored in the storage unit 300.

Then, the inter-frame signal strength correlation value 330 (refer to FIGS. 5A, 5B, and 11) is calculated from the reflected wave data 320 (step S22). Then, an ultrasonic transducer from which a correlation value of a minimum value equal to or less than a predetermined reference value is obtained is determined to be a scanning line immediately above the blood vessel, and a scanning line ID corresponding to the ultrasonic transducer is registered in the list of scanning lines immediately above a blood vessel 340 (refer to FIG. 11) (step S24). Then, the process of detecting the scanning lines immediately above the blood vessel is ended.

FIG. 14 is a flowchart for explaining the flow of the process of detecting the vascular wall depth position candidate in the ultrasonic measurement apparatus 10 according to the present embodiment.

In this process, the processing unit 200 extracts a local peak, in which the signal strength satisfies the predetermined vascular wall equivalent signal level Pw1 (refer to FIGS. 5A and 5B), from the reflected wave data 320 of the scanning line for each scanning line immediately above the blood vessel that is registered in the list of scanning lines immediately above a blood vessel 340, thereby generating the signal strength peak list 350 for each scanning line immediately above the blood vessel (step S40). Then, peaks of the signal strength equal to or less than the minimum reference depth Ld are excluded from the list (step S42), and the process of detecting the vascular wall depth position candidate is ended.

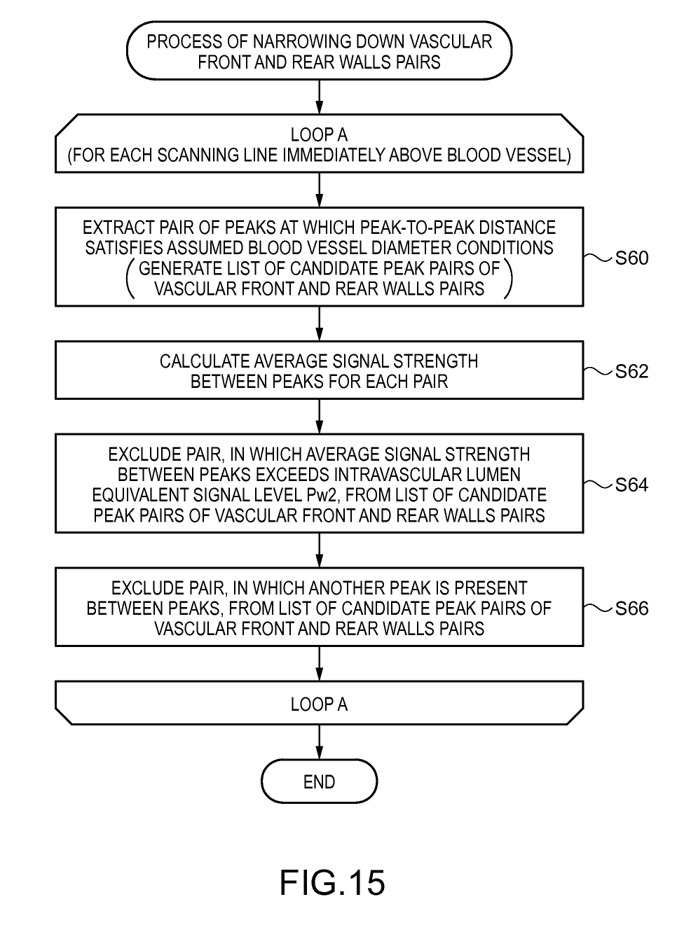

FIG. 15 is a flowchart for explaining the flow of the process of narrowing down the vascular front and rear walls pairs in the ultrasonic measurement apparatus 10 according to the present embodiment.

In this process, the processing unit 200 executes loop A for each scanning line immediately above the blood vessel that is registered in the list of scanning lines immediately above a blood vessel 340 (steps S60 to S66).

In the loop A, first, the processing unit 200 generates a pair from the registered peaks with reference to the signal strength peak list 350 corresponding to the scanning lines immediately above the blood vessel to be processed, and extracts a pair in which a peak-to-peak distance satisfies predetermined assumed blood vessel diameter conditions, thereby generating the list of candidate peak pairs of vascular front and rear walls pairs 360 (step S60). The assumed blood vessel diameter conditions referred to herein are conditions defining a rough range of the blood vessel diameter suitable for the measurement, and it is assumed that the assumed blood vessel diameter conditions are set in advance by testing or the like.

Then, an average signal strength between peaks is calculated for each pair of peaks registered in the list of candidate peak pairs of vascular front and rear walls pairs 360 (step S62), and a pair in which the average signal strength between peaks exceeds the intravascular lumen equivalent signal level Pw2 (refer to FIG. 6C) is excluded from the list of candidate peak pairs of vascular front and rear walls pairs 360 (step S64).

In addition, among the peaks registered in the list of candidate peak pairs of vascular front and rear walls pairs 360, a pair in which another peak is present between peaks is excluded from the list (step S66), and the loop A is ended. The pair of peaks remaining in the list of candidate peak pairs of vascular front and rear walls pairs 360 in this stage is front and rear walls of the blood vessel in the scanning lines immediately above the blood vessel to be processed.

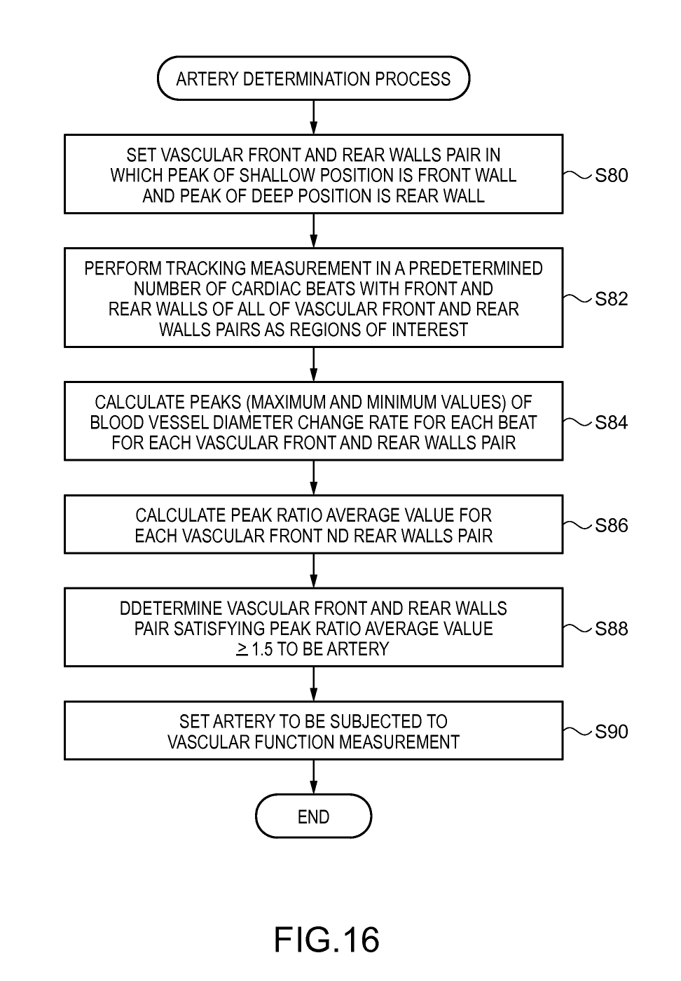

FIG. 16 is a flowchart for explaining the flow of the artery determination process in the ultrasonic measurement apparatus 10 according to the present embodiment.

In this process, first, for each pair of peaks remaining in the list of candidate peak pairs of vascular front and rear walls pairs 360, the processing unit 200 generates the vascular front and rear walls pair data 380 (refer to FIG. 12) by regarding the peak of the relatively shallow position (position where the depth from the body surface is small) of the pair as a front wall and the peak of the relatively deep position as a rear wall (step S80).

Then, the processing unit 200 sets the front wall signal strength peak depth 381 and the rear wall signal strength peak depth 382 of all pieces of the vascular front and rear walls pair data 380 as a region of interest, and tracks the displacement of each region of interest in a predetermined number of cardiac beats (step S82). It is also possible to use the reflected wave data 320 that is already stored. Then, for each vascular front and rear walls pair, the peak of the blood vessel diameter change rate is calculated for each beat of the cardiac cycle, thereby generating the diameter change rate peak history data 383 (step S84).

Then, the processing unit 200 calculates the peak ratio average value 387 for each vascular front and rear walls pair (step S86). Then, the processing unit 200 determines a vascular front and rear walls pair, which has a peak ratio equal to or greater than a predetermined threshold value (in the present embodiment, 1.5), to be an artery and sets the artery determination flag 388 to “1”, and determines a vascular front and rear walls pair having a peak ratio less than the predetermined threshold value and sets the artery determination flag 388 to “0” (step S88). Then, the processing unit 200 sets a vascular front and rear walls pair having the largest peak ratio average value 387 as a target artery for vascular function measurement (step S90), and the artery determination process is ended.

As described above, according to the present embodiment, it is possible to find an artery automatically from the tissues in the body in the scanning range As (refer to FIG. 3) of the ultrasonic probe 16 and to perform vascular function measurement with the artery as a measurement target. Therefore, since the only thing that the operator has to do is to place the ultrasonic probe 16 at an approximate place where the carotid artery may be present, a labor in the measurement work is greatly reduced. As a result, measurement errors can also be reduced significantly.

### Modification Examples

In addition, embodiments of the invention are not limited to the embodiment described above, and constituent components can be appropriately added, omitted, and changed.

### (A) Detection of a Depth Position Candidate of a Blood Vessel

In addition, a vessel wall depth position candidate may be detected by performing a correlation operation of the received signal strength. Specifically, the depth position of a blood vessel is detected by calculating the degree of change in the received signal strength by performing an autocorrelation operation of the received signal strength between different frames for each depth position.

Detailed explanation will be given below.

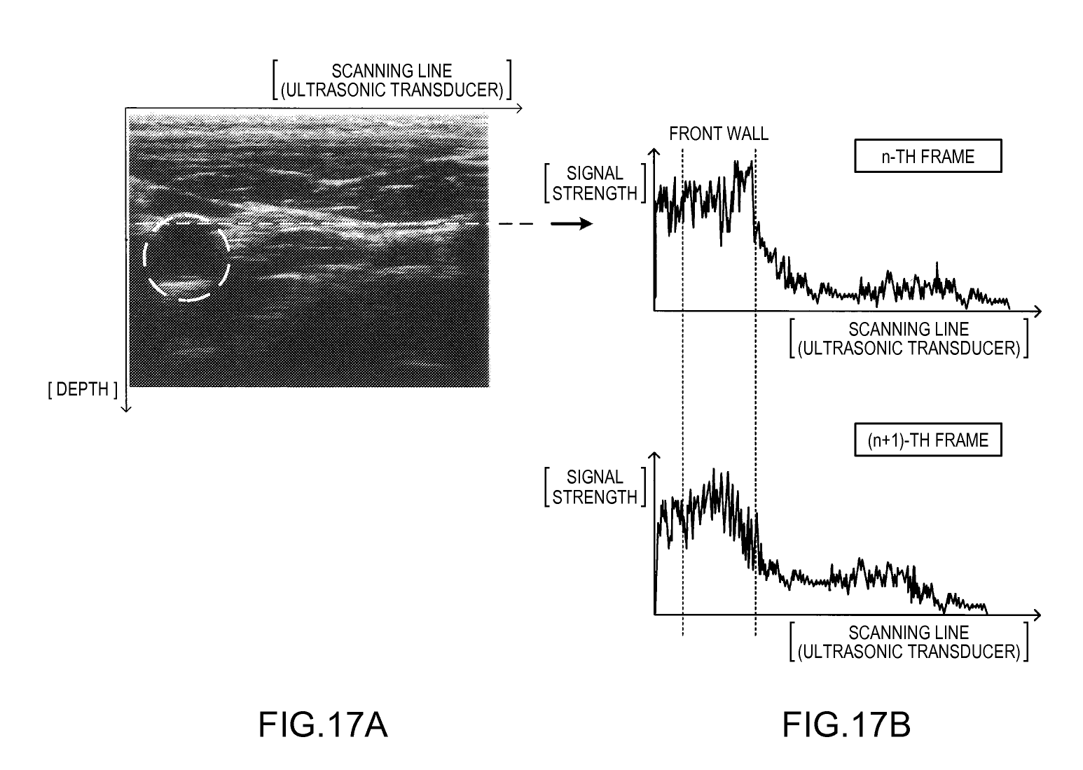

FIGS. 17A and 17B are diagrams illustrating the received signal strength at a certain depth position. FIG. 17A shows a B-mode image obtained from the measurement result of each ultrasonic transducer, and FIG. 17B shows a “scanning line-signal strength graph” obtained from the B-mode image shown in FIG. 17A. FIG. 17B shows a graph in each of first and second frames. The “scanning line-signal strength graph” is a graph showing the received signal strength along a scanning line direction for a certain depth position. As described above, if there is a blood vessel, a strong reflected wave applied to the front wall or the rear wall is detected. In FIG. 17B, a strong reflected wave relevant to the front wall of the blood vessel appears. When the received signal strengths are compared between frames, a change in the signal strength occurs at a scanning line position corresponding to the blood vessel position.

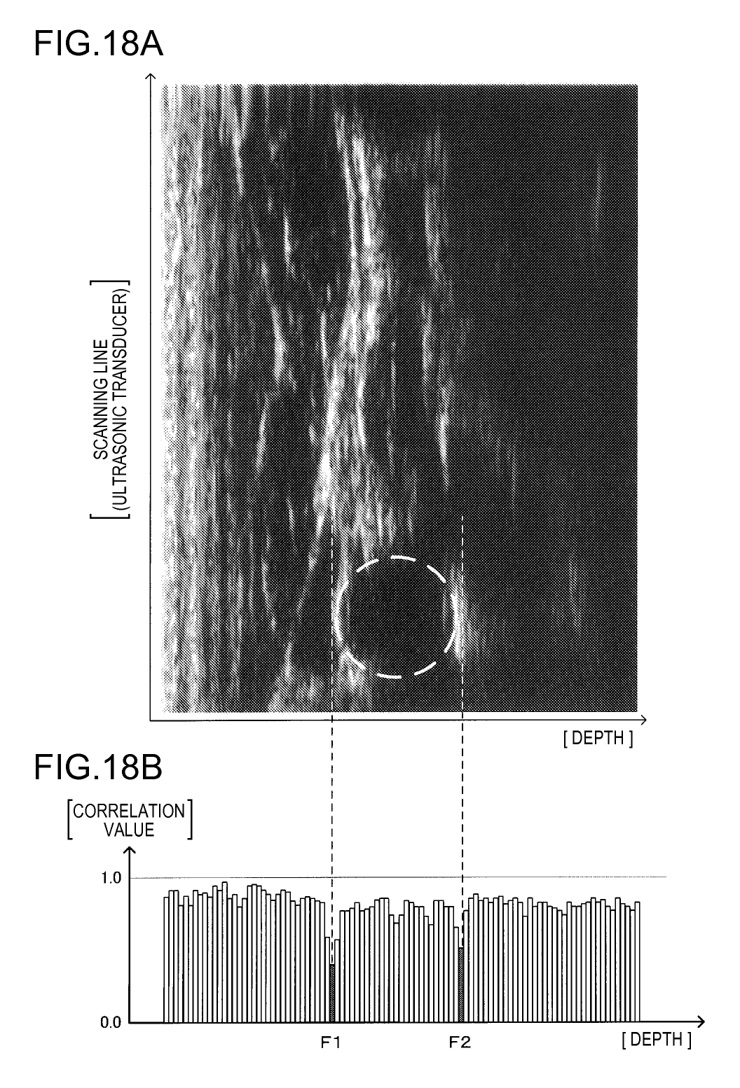

FIGS. 18A and 18B are diagrams illustrating the detection of the depth position of a blood vessel based on the correlation operation of the signal strength between frames. FIG. 18A is a B-mode image, and FIG. 18B is a histogram showing the correlation value of the received signal strength at each depth position between two consecutive frames. The correlation value is a value normalized in the range of “0.0 to 1.0”.

When there is no blood vessel at a certain depth position, the correlation value of the received signal strength graph between two consecutive frames is large because there is almost no change in the received signal strength. On the other hand, when there is a blood vessel, the correlation value is small because the received signal strength of a portion corresponding to the scanning line position of the blood vessel is changed. As described above, the received signal strength is a measurement result according to an ultrasonic transducer, and the reflected wave from the front and rear walls of the blood vessel is detected strongly, but the reflected wave reflected from the lateral wall is weak. That is, as shown in the histogram of FIG. 18B, the correlation value at the depth positions of the front and rear walls of the blood vessel is small. Therefore, in the histogram of FIG. 18B, depth positions at which the correlation value corresponds to the minimum value is determined to be the depth positions of the front and rear walls of the blood vessel. In FIGS. 18A and 18B, depth positions F1 and F2 correspond to the depth positions of the front and rear walls of the blood vessel.

The entire disclosure of Japanese Patent Application No. 2014-046557, filed on Mar. 10, 2014 is expressly incorporated by reference herein.

What is claimed is:

1. An ultrasonic measurement apparatus, comprising:
a transmission and reception control unit that controls transmission of an ultrasonic wave to a blood vessel and reception of the ultrasonic wave that is reflected from the blood vessel;
a vessel wall detection unit that detects first and second walls of the blood vessel based on a received signal obtained from the received ultrasonic wave; and
a blood vessel determination unit that determines the blood vessel based on a distance between the first and second walls.

2. The ultrasonic measurement apparatus according to claim 1, further comprising:
a unit that detects a position immediately above the blood vessel using a correlation value obtained by autocorrelation of the received signal.

3. The ultrasonic measurement apparatus according to claim 1, further comprising:
a depth position detection unit that detects a depth position of the blood vessel using a correlation value obtained by autocorrelation of the received signal at different depth positions.

4. The ultrasonic measurement apparatus according to claim 1,
wherein the blood vessel determination unit determines a type of the blood vessel using a temporal change in an increasing direction of the distance and a temporal change in a decreasing direction of the distance.

5. The ultrasonic measurement apparatus according to claim 1, further comprising:
a vascular function measuring unit that performs vascular function measurement of the blood vessel by continuing position measurement with the first and second walls of the blood vessel as tracking targets when the blood vessel is determined to be an artery by the blood vessel determination unit.

6. An ultrasonic measurement method, comprising:
controlling transmission of an ultrasonic wave to a blood vessel and reception of the ultrasonic wave that is reflected from the blood vessel;
detecting first and second walls of the blood vessel based on a received signal obtained from the received ultrasonic wave; and
determining the blood vessel based on a distance between the first and second walls.
